(19) United States
(12) Patent Application Publication
(10) Pub. No.: US 2024/0006026 A1
(43) Pub. Date: Jan. 4, 2024

(54) GENOME ASSEMBLY METHOD, APPARATUS, DEVICE AND STORAGE MEDIUM

(71) Applicant: SUN YAT-SEN UNIVERSITY, Guangzhou (CN)

(72) Inventors: Yutong LU, Guangzhou (CN); Ying WANG, Guangzhou (CN); Zhiguang CHEN, Guangzhou (CN)

(73) Assignee: SUN YAT-SEN UNIVERSITY, Guangzhou (CN)

(21) Appl. No.: 18/454,977

(22) Filed: Aug. 24, 2023

Related U.S. Application Data
(63) Continuation of application No. PCT/CN2022/100178, filed on Jun. 21, 2022.

(30) Foreign Application Priority Data
Mar. 28, 2022 (CN) 202210311761.3

(51) Int. Cl. G16B 30/20; G16B 40/30

(52) U.S. Cl. CPC G16B 30/20; G16B 40/30

(57) ABSTRACT

Disclosed are a genome assembly method, a genome assembly apparatus, a device and a storage medium. The method includes: obtaining a gene short sequence, and determining a first segmentation value; segmenting the gene short sequence based on the first segmentation value to obtain each gene subsequence; globally sorting each gene subsequence based on a preset grouped parallel sorting by regular sampling to obtain each sorted gene subsequence; traversing the distributed gene map in parallel to obtain each continuous gene sequence, and filling and assembling each continuous gene sequence to obtain each target continuous gene sequence; and determining a second segmentation value, and in response to that the second segmentation value is greater than or equal to a preset maximum segmentation threshold, assembling each target continuous gene sequence to obtain a genome assembly result.

### CROSS-REFERENCE TO RELATED APPLICATIONS

This application is a continuation application of International Application No. PCT/CN2022/100178, filed on Jun. 21, 2022, which claims priority to Chinese Patent Application No. 202210311761.3, filed on Mar. 28, 2022. The disclosures of the above-mentioned applications are incorporated herein by reference in their entireties.

### TECHNICAL FIELD

The present application relates to the technical field of genome assembly, in particular to a genome assembly method, a genome assembly apparatus, a device and a storage medium.

### BACKGROUND

Existing genome assembly algorithms for assembling next-generation sequencing data mainly use the De Bruijn graph structure. In order to improve the efficiency of genome assembly, the parallel sorting by regular sampling is usually used to sort the De Bruijn graph structure. However, as the number of processes increases, the number of sampling points for each process also increases, and the number of sampling points for the entire algorithm increases quadratically. Besides, when traversing the existing genome assembly algorithm, each process must randomly select a vertex as the seed of the gene segment where it is located, and extend forward and backward in two directions to find the complete gene segment, which may happen that different initial vertices selected by two processes belong to the same gene segment, Moreover, with the gradual outward extension, the vertices passed by a gene segment are scattered in a large number of processes, and the computational complexity is high, which in turn leads to low genome assembly efficiency.

### SUMMARY

The main purpose of the present application is to provide a genome assembly method, a genome assembly apparatus, a device and a storage medium, aiming to solve the technical problem of high computational complexity of genome assembly, resulting in low assembly efficiency in the related art.

In order to achieve the above objective, the present application provides a genome assembly method, including:
obtaining a gene short sequence, and determining a first segmentation value;
segmenting the gene short sequence based on the first segmentation value to obtain each gene subsequence;
globally sorting each gene subsequence based on a preset grouped parallel sorting by regular sampling to obtain each sorted gene subsequence;
constructing a distributed gene map based on each sorted gene subsequence;
traversing the distributed gene map in parallel to obtain each continuous gene sequence, and filling and assembling each continuous gene sequence to obtain each target continuous gene sequence; and
determining a second segmentation value, and in response to that the second segmentation value is greater than or equal to a preset maximum segmentation threshold, assembling each target continuous gene sequence to obtain a genome assembly result.

The present application further provide a genome assembly apparatus, including:
an acquisition module configured for obtaining a gene short sequence, and determining a first segmentation value;
a segmentation module configured for segmenting the gene short sequence based on the first segmentation value to obtain each gene subsequence;
a global sorting module configured for globally sorting each gene subsequence based on a preset grouped parallel sorting by regular sampling to obtain each sorted gene subsequence;
a construction module configured for constructing a distributed gene map based on each sorted gene subsequence;
a parallel traversal module configured for traversing the distributed gene map in parallel to obtain each continuous gene sequence, and filling and assembling each continuous gene sequence to obtain each target continuous gene sequence; and
an assembly module configured for determining a second segmentation value, and in response to that the second segmentation value is greater than or equal to a preset maximum segmentation threshold, assembling each target continuous gene sequence to obtain a genome assembly result.

The present application further provides a vehicle-mounted device for genome assembly. The vehicle-mounted device for genome assembly is an entity device, and includes a memory, a processor, and a genome assembly program stored in the memory. When the genome assembly program is executed by the processor, the genome assembly method as described above is implemented.

The present application further provides a storage medium. The storage medium is a computer-readable storage medium. A genome assembly program is stored in the computer-readable storage medium, and when the genome assembly program is executed by a processor, the genome assembly method as described above is implemented.

The present application provides a genome assembly method, a genome assembly apparatus, a device and a storage medium. The genome assembly method includes: obtaining a gene short sequence, and determining a first segmentation value; segmenting the gene short sequence based on the first segmentation value to obtain each gene subsequence; globally sorting each gene subsequence based on a preset grouped parallel sorting by regular sampling to obtain each sorted gene subsequence; constructing a distributed gene map based on each sorted gene subsequence; traversing the distributed gene map in parallel to obtain each continuous gene sequence, and filling and assembling each continuous gene sequence to obtain each target continuous gene sequence; and determining a second segmentation value, and in response to that the second segmentation value is greater than or equal to a preset maximum segmentation threshold, assembling each target continuous gene sequence to obtain a genome assembly result. Thus, the global sorting based on the preset grouped parallel sorting by regular sampling is realized, such that the number of samples per process is reduced from the original number of system processes to the number of groups, thereby greatly reducing the number of sampling points of the system that increases quadratically due to the increase in the number of processes. In turn, the time complexity of parallel sorting is reduced, and the distributed gene map is traversed in parallel to generate continuous gene sequences, which effectively improves the efficiency of genome assembly.

### BRIEF DESCRIPTION OF THE DRAWINGS

The accompanying drawings, which are incorporated in and constitute a part of this specification, illustrate embodiments consistent with the present application and together with the description serve to explain the principles of the present application.

In order to more clearly illustrate the technical solutions in the embodiments of the present application or the related art, the following will briefly introduce the drawings that need to be used in the description of the embodiments or the related art. Obviously, for those skilled in the art, other drawings can also be obtained based on these drawings without any creative effort.

FIG. 1 is a schematic flowchart of a genome assembly method according to a first embodiment of the present application.

FIG. 2 is a schematic diagram of a business process of the genome assembly method of the present application.

FIG. 3 is a schematic flowchart of the genome assembly method according to a second embodiment of the present application.

FIG. 4 is a schematic flowchart of a grouped parallel sorting by regular sampling in the genome assembly method of the present application.

FIG. 5 is a schematic flowchart of the genome assembly method according to a third embodiment of the present application.

FIG. 6 is a schematic flowchart of parallel traversal of the distributed gene map in the genome assembly method of the present application.

FIG. 7 is a schematic structural diagram of a vehicle-mounted device for genome assembly in the hardware operating environment according to an embodiment of the present application.

The realization of the objective, functional characteristics, and advantages of the present application are further described with reference to the accompanying drawings.

### DETAILED DESCRIPTION OF THE EMBODIMENTS

It should be understood that the specific embodiments described herein are only used to explain the present application, not to limit the present application.

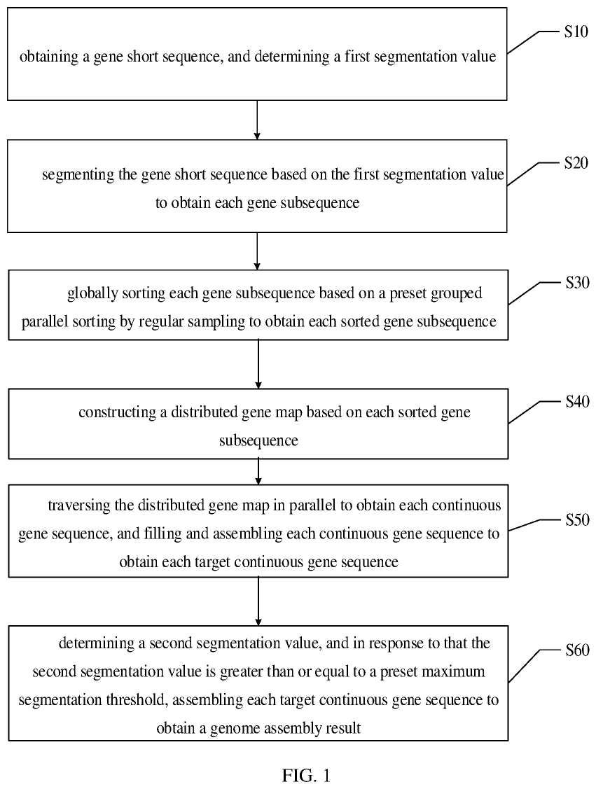

As shown in FIG. 1, FIG. 1 is a schematic flowchart of a genome assembly method according to a first embodiment of the present application. The genome assembly method includes:

Operation S10, obtaining a gene short sequence, and determining a first segmentation value.

In an embodiment, it should be noted that sequencing of genomic fragments produces random reads that are distributed randomly across the genome. The process of genome assembly is to arrange and connect these reads in the correct order, assemble them into DNA fragments (contig) with continuous bases, and finally restore the sequence of the entire chromosome and the entire genome.

In an embodiment, the sequencing data files in fasta or fastq format are read in parallel through each process of the system to obtain the gene short sequence, and then the first separation value in the preset candidate window sequence is selected according to the preset selection rules. The preset candidate window sequence is manually defined. The numerical range in the preset candidate window sequence is greater than 21 and less than 99. The preset selection rule is to select the smallest numerical value in the preset candidate window sequence or directly rank the first value in the preset candidate window sequence as the first separation value.

Operation S20, segmenting the gene short sequence based on the first segmentation value to obtain each gene subsequence.

In an embodiment, the first segmentation value is added to a preset maximum segmentation threshold to obtain a segmentation window. The preset maximum segmentation threshold is a positive integer greater than 0. The preset maximum segmentation threshold is set to 1. The segmentation window is the window size for segmenting the gene short sequence, and then based on the segmentation window, the gene short sequence is scanned and segmented to obtain each gene subsequence. For example, the gene short sequence is ACTAGCTA, the first segmentation value is 2, and the preset maximum segmentation threshold is 1, and then the gene subsequences obtained by segmentation are ACT, CTA, TAG, GCT and CTA.

Operation S30, globally sorting each gene subsequence based on a preset grouped parallel sorting by regular sampling to obtain each sorted gene subsequence.

In this embodiment, a prefix sequence corresponding to the first segmentation value in each gene subsequence is reversed and sorted in alphabetical order, and each gene subsequence is sorted based on the sorting result to obtain each initial sorting sequence. Then the processes on the system are grouped, and then through the grouped processes, regular sampling sorting are performed on each initial sorting sequence in parallel to obtain each sorted gene subsequence. Therefore, by grouping the processes in the system, the sampling number of each process is reduced from the original number of system processes to the number of groups, which not only reduces memory overhead, but also reduces the sorting time of sampling points. In addition, by using packet communication instead of global communication, the synchronization waiting time is effectively reduced.

In an embodiment, the above operation S30: the globally sorting each gene subsequence based on the preset grouped parallel sorting by regular sampling to obtain each sorted gene subsequence includes:

reversing a prefix sequence corresponding to the first segmentation value in each gene subsequence and sorting in alphabetical order, and sorting each gene subsequence based on the sorting result to obtain each initial sorting sequence.

In this embodiment, the method includes: determining that the prefix length in each gene subsequence is the prefix sequence corresponding to the first segmentation value, reversing the prefix sequence in each gene subsequence, sorting the reversed prefix sequences in alphabetical order to obtain the sorting results of each prefix sequence, and sorting the gene subsequences based on the sorting results to obtain the initial sorting sequences. For example, the first segmentation value k is 6, and the three (k+1)-mer gene subsequences are ACTAGCT, CTGAGCC, GTATGGA, and ACTTGGA, the k-mer prefix sequences whose prefix length is the first segmentation value are ACTAGC, CTGAGC, GTATGG, and ACTTGG, and the reversed prefix sequence is CGATCA, CGAGTC, GGTATG, and GGTTCA. Thus, after the reversal, the tail of the k-mer goes to the high position of the code, and then the reversed prefix sequences are sorted in alphabetical order and expressed as CGAGTC, CGATCA, GGTATG and GGTTCA, the corresponding (k+1)-mer gene subsequences are sorted as CTGAGCC, ACTAGCT, GTATGGA, and ACTTGGA, so that (k+1)-mers with similar prefix k-mer tails are stored closer together, thereby improving the subsequent traversal efficiency.

The above operation S30 further includes: obtaining a number of processes, and grouping each process based on the number to obtain each process group, each process in each process group being provided with a corresponding number.

In this embodiment, it should be noted that each of the processes is grouped, and each group has its corresponding group number. Each process in each group is provided with a corresponding number, for example, group 0, group 1, group 2, etc., and group 0 includes process 0, process 1, process 2, and so on.

The above operation S30 further includes: using each initial sorting sequence as an element to be sorted, and assigning each element to be sorted to each process.

In this embodiment, an initial sorting sequence is taken as an element to be sorted, and then each of the elements to be sorted is equally assigned to each of the processes. For example, there are n elements to be sorted, and there are p processes in the system, and each process is responsible for processing w=n/p elements.

The above operation S30 further includes: performing sorting by regular sampling on each element to be sorted in parallel through each process in each process group to obtain each sorted gene subsequence.

In this embodiment, the method includes: sorting each element to be sorted in each of the processes to obtain a first sorting element, and performing regular sampling on the first sorting element based on the number of the process group to obtain a first sampled element; sending the first sampled element in each process to a first numbered process of the corresponding process group, for the first numbered process in each process group, sorting and performing regular sampling on each first sampled element in parallel to obtain group sampling elements of each process group; sending each group sampling element to a preset global process, and sorting and performing regular sampling on each group sampling element through the preset global process to obtain a global sampling element; dividing the first sorting element in each process based on the global sampling element to obtain each division element, and recording number of elements and displacements corresponding to each division element; forming each process group with the same number between different process groups as a new communication subdomain; for each process in each communication subdomain, based on the number of elements and displacements corresponding to each division element in each process, performing data exchange on each division element in each process to obtain a target element in each process; merging and sorting the target element in each process to obtain a second sorting element; and performing sorting by regular sampling on the second sorting element of each process in each communication subdomain in parallel to obtain each sorted gene subsequence. Thus, by grouping the processes in the system, the sampling number of each process is reduced from the original number of system processes to the number of groups, which not only reduces memory overhead, but also reduces the sorting time of sampling points. In addition, by using packet communication instead of global communication, the synchronization waiting time is effectively reduced.

Operation S40, constructing a distributed gene map based on each sorted gene subsequence.

In this embodiment, it should be noted that the distributed gene map is a De Bruijn graph of distributed storage. In the related art, a hash method is usually used to construct a De Bruijn graph, and an ordered array is used. However, in the present application, the gene subsequences are globally sorted, and the graph is constructed in the form of an ordered array, which is conducive to improving communication efficiency. After the sorted gene subsequences are obtained, the same sorted gene subsequences are combined, thereby counting the frequency of each sorted gene subsequence. Further, the sorted gene subsequences whose frequency is lower than the preset frequency threshold are filtered, and each target sorted gene subsequence whose frequency exceeds the preset frequency threshold is retained. Finally, each of the target sorted gene subsequences is used as the edge of the graph, and the sequence corresponding to the first segmentation value in each of the target sorted gene subsequences is used as the vertex of the distributed gene map. The sequence whose prefix length is the first segmentation value indicates that the target sorted gene subsequence is selected to be the first sequence and the length is equal to the sequence corresponding to the first segmentation value. For example, the first segmentation value is k, and multiple (k+1)-mers are obtained by splitting. After sorting and filtering, the retained (k+1)-mers are used as edges to build the graph. Furthermore, in (k+1)-mers, the sequence whose prefix length is equal to k is selected as the vertex of the graph.

Operation S50, traversing the distributed gene map in parallel to obtain each continuous gene sequence, and filling and assembling each continuous gene sequence to obtain each target continuous gene sequence.

In this embodiment, it should be noted that, before traversal, it is necessary to detect special structures caused by sequencing errors to correct errors, for example, short, low coverage, dead ends, bubble structures, false links and other sequences.

The distributed gene map is traversed by preset graph-coloring-based hierarchical parallel depth-first search algorithm. The hierarchical parallel depth-first search algorithm is a method of single-point traversal after merging the paths of the distributed gene map. The vertices whose in-degree or out-degree is 0 in the distributed gene map are colored, and a preset number of vertices are randomly selected for coloring to obtain each coloring point. Each coloring point is taken as the start point, the distributed gene map is traversed in parallel, and the depth search is stopped until the next coloring vertex is accessed, thereby effectively reducing the depth of a single depth-first search, and then merging and saving the intermediate path between the two coloring points to obtain the merged distributed gene map, then the space complexity of the depth-first traversal of the merged distributed gene map is calculated, and based on the space complexity, it is determined whether the graph scale of the merged distributed gene map meets the preset requirements. If the graph scale of the merged distributed gene map meets the preset requirements, then single-point depth-first traversal is performed on the merged distributed gene map to obtain the target traversal paths between the vertices whose in-degree is 0 to the vertices whose out-degree is 0. Since there may be a merged intermediate path in the target traversal path, the target traversal path is traced back to obtain each target specific path, and finally each continuous gene sequence is determined based on each target specific path.

Further, a set of gene sequences (each continuous gene sequence in this example) is obtained after traversal, it is called contigs. However, in the process of correcting the errors of each continuous gene sequence, there may be a situation where the gene sequence is disconnected by mistaken deletion, which is manifested as a gap between contigs. To make the assembled sequence more complete, these contigs need to be filled.

The following operations are performed for each continuous gene sequence. Aligning the continuous gene sequence contigs with each gene short sequence. If there is a gene short sequence that can be aligned with the head or tail of the continuous gene sequence contig, the paired-end of the gene short sequence is added to the continuous gene sequence. It should be noted that paired-end means paired-end sequencing: it means that when the DNA library to be tested is constructed, the sequencing primer binding sites are added to the adapters at both ends. After the first round of sequencing is completed, the template strand of the first round of sequencing is removed, and the paired-end module is used to guide the regeneration and amplification of the complementary strand in situ, to achieve the amount of template used in the second round of sequencing, and perform the second round of complementary chain synthesis sequencing. In this way, the length of the continuous gene sequence is extended to obtain the target continuous gene sequence, which helps to restore the gene subsequence that has been deleted by mistake, and also helps to reconstruct some gene sequences with low repetition.

Operation S60, determining a second segmentation value, and in response to that the second segmentation value is greater than or equal to a preset maximum segmentation threshold, assembling each target continuous gene sequence to obtain a genome assembly result.

In this embodiment, the next value in the preset candidate window sequence is used as the second segmentation value, and then it is determined whether the second segmentation value satisfies the preset maximum segmentation threshold. The preset maximum segmentation threshold is the threshold of the maximum window of the segmented gene short sequence, and the preset maximum segmentation threshold can be set to 99. If the second segmentation value is greater than the preset maximum segmentation threshold, each target continuous gene sequence is assembled to obtain a genome assembly result.

After the determining the second segmentation value, the genome assembly method further includes:
in response to that the second segmentation value is less than the preset maximum segmentation threshold, extracting each segmentation sequence from each target continuous gene sequence based on the second segmentation value, and segmenting the gene short sequence based on the second segmentation value to obtain each gene subsequence until obtaining each new sorted gene subsequence;
merging each segmentation sequence and each new sorted gene subsequence to obtain each merged gene sequence;
constructing the distributed gene map based on each merged gene sequence; and
traversing the distributed gene map in parallel to obtain each continuous gene sequence, and filling and assembling each continuous gene sequence to obtain each target continuous gene sequence until the determined segmentation value is greater than the preset maximum segmentation threshold, assembling each new target continuous gene sequence to obtain the genome assembly result.

In this embodiment, it should be noted that if the second segmentation value is smaller than the preset maximum segmentation threshold, based on the second segmentation value, adding the second segmentation threshold to a preset segmentation value, extracting each segmentation sequence from the target continuous gene sequence, segmenting the gene short sequence based on the second segmentation threshold, and then globally sorting the segmented gene subsequence. The segmentation process and the sorting process are basically the same as the specific implementation scheme of operation S20 to operation S30, and will not be repeated herein. Further, after obtaining the sorted gene subsequences corresponding to the global sorting, merging each segmentation sequence and each new sorted gene subsequence to obtain each merged gene sequence. Furthermore, based on each of the merged sequences, a distributed gene map is constructed. The map construction process is basically the same as the implementation of operation S40, and will not be repeated herein. Then, traversing the distributed gene map in parallel to obtain each continuous gene sequence, and filling and assembling each continuous gene sequence to obtain each target continuous gene sequence until the determined segmentation value is greater than the preset maximum segmentation threshold, assembling each new target continuous gene sequence to obtain the genome assembly result.

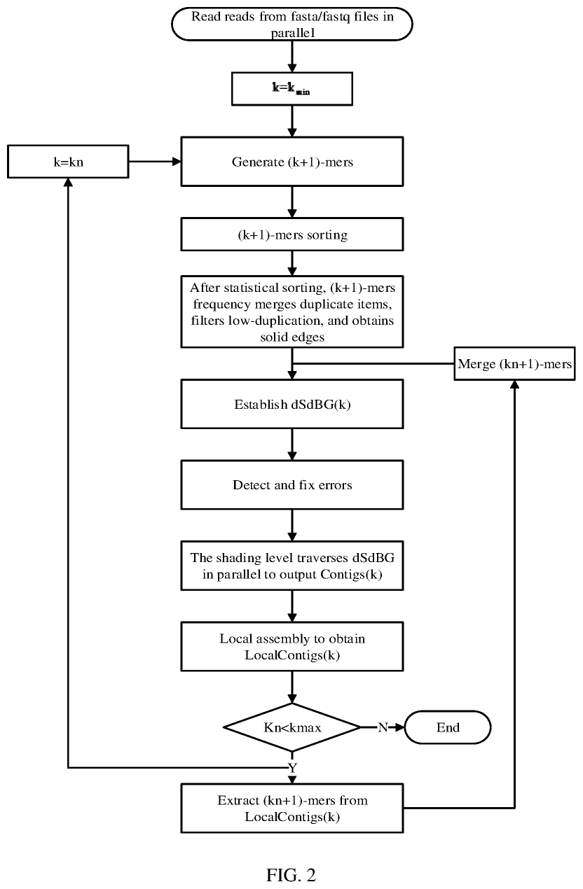

Further, as shown in FIG. 2, FIG. 2 is a schematic diagram of a business process of the genome assembly method of the present application. reads is the gene short sequence, kmin is the minimum value in the preset candidate window sequence, k is the first segmentation value, (k+1)mers is each gene subsequence, (k+1)mers sorting is the global sorting of each gene subsequence based on the preset grouped parallel sorting by regular sampling, the sorted (k+1)mers are each sorted gene subsequence, the solid edge is the target sorted gene subsequence, dSdBG(k) is the distributed gene map, the coloring hierarchical parallel DFS traverses the preset hierarchical parallel depth-first search algorithm based on graph coloring, Contigs(k) is the continuous gene sequence, LocalContigs(k) is the target continuous gene sequence, kn is the second segmentation value, kmax is the preset maximum segmentation threshold, and (kn+1)-mers is the segmentation sequence extracted from the target continuous gene sequence.

In an embodiment, reading the gene short sequence in parallel to select the minimum value in the preset candidate window sequence as the first segmentation value, then using (the first segmentation value+1) as the segmentation window to segment the gene short sequence to obtain each gene subsequence. Based on a preset grouped parallel sorting by regular sampling, sorting the gene subsequences to obtain each sorted gene subsequence. Counting the frequency of each sorted gene subsequence and merging each sorted gene subsequence, and then filtering out the sorted gene subsequences whose frequency is lower than the preset frequency threshold, to obtain the final target sorted gene subsequence, thereby establishing the distributed gene map based on the target sequenced gene subsequence. Further, traversing the distributed gene map through a preset hierarchical parallel depth-first search algorithm based on graph coloring to obtain each continuous gene sequence, and then filling and assembling each continuous gene sequence to obtain each target continuous gene sequence. Further, determine the second segmentation value in the preset candidate window sequence, if the second segmentation value is greater than or equal to the preset maximum segmentation threshold, then assemble each target continuous gene sequence to obtain a genome assembly result. If the second segmentation value is less than the preset maximum segmentation threshold, extract each segmentation sequence whose size is 1 (second segmentation value+1) from each target continuous gene sequence, and return to the operation of segmenting the gene short sequence to re-segment through the new segmentation value until the new target sequenced gene subsequence is obtained, thereby merging each of the new target sorted gene subsequences and each segmentation sequence to construct a new distributed gene map. Thus, the operations of traversal and filling assembly are continued until the segmentation value selected in the preset candidate window sequence is greater than or equal to the preset maximum segmentation threshold, so as to obtain the final genome assembly result.

In an embodiment of the present application, through the above solutions, the global sorting based on the preset group parallel sorting by regular sampling is realized, so that the sampling number of each process is reduced from the original number of system processes to the number of groups, thereby greatly reducing the number of system sampling points that increases quadratically due to the increase in the number of processes. In turn, the time complexity of parallel sorting is reduced, and the distributed gene map is traversed in parallel to generate continuous gene sequences, which effectively improves the efficiency of genome assembly.

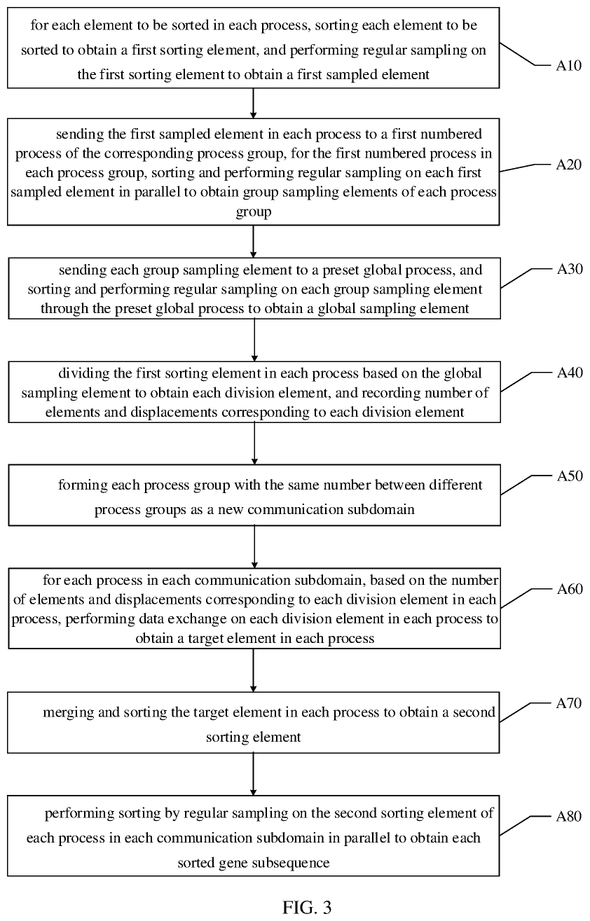

As shown in FIG. 3, based on the first embodiment of the present application, in another embodiment of the present application, the operation of performing sorting by regular sampling on each element to be sorted in parallel through each process in each process group to obtain each sorted gene subsequence includes:

Operation A10, for each element to be sorted in each process, sorting each element to be sorted to obtain a first sorting element, and performing regular sampling on the first sorting element to obtain a first sampled element.

In this embodiment, it should be noted that, an element to be sorted represents a gene subsequence. The following operations are performed for each element to be sorted in each process:

performing quick sorting on each of the elements to be sorted, and then using each element obtained by the quick sorting as the first sorting element. Further, regular sampling is performed on the first sorting element to obtain the first sampling element. It should be noted that the number of elements sampled is related to the number of process groups. For example, if the processors of each process are divided into g groups, and each group has q processors, then g−1 elements are sampled.

Operation A20, sending the first sampled element in each process to a first numbered process of the corresponding process group, for the first numbered process in each process group, sorting and performing regular sampling on each first sampled element in parallel to obtain group sampling elements of each process group.

In this embodiment, it should be noted that since each process of each process group is provided with its corresponding number, for example, each process group has 4 processes, and the process numbers can be set as process 0, process 1, process 2 and process 3, etc.

In an embodiment, the following operations are performed for each process group:
sending the first sampling element sampled by each process in the process group to the first numbered process in the process group, merging and sorting each of the first sampling elements, and then performing regular sampling on each of the sorted first sampling elements to obtain the group sampling elements of the process group. For example, following the example of operation A10 above, sending the first sampling elements sampled by each process to process 0, that is, the number of elements obtained by process 0 is q*(g−1). Further, the collected q*(g−1) elements are merged and sorted, and the sorting elements are regularly sampled (g−1) elements as the group sampling elements.

Operation A30, sending each group sampling element to a preset global process, and sorting and performing regular sampling on each group sampling element through the preset global process to obtain a global sampling element.

In this embodiment, following the above example, process 0 in each group sends g−1 group sampling elements to global process 0, the global process 0 collects the group sampling elements of the process 0 in all groups, a total of g*(g−1) group sampling elements. Further, each group of sampling elements is sorted, and (g−1) group sampling elements are regularly sampled for each sorted group of sampling elements to obtain a global sampling element.

Operation A40, dividing the first sorting element in each process based on the global sampling element to obtain each division element, and recording number of elements and displacements corresponding to each division element.

In this embodiment, it should be noted that, the displacement is the offset of the division element in the first sorting element. Globally broadcast global sampling elements by preset global process, according to the global sampling elements, respectively divide the first sorting elements in each of the processes to obtain each division element, and record the number of elements and displacement corresponding to each division element. For example, the global sampling elements are (g−1) elements, and the first sorting elements are divided into g parts. It should be noted that the global sampling element is the division standard for dividing the local first sorting element of the process into g parts. For example, the global sampling element has (g−1) sampling elements, based on each element in the first sorting element, the elements smaller than the first sampling element are divided into the first part, and the elements larger than the first sampling element and smaller than the second sampling element are divided into the second part. By analogy, the first sorting element can be divided into g parts.

Operation A50, forming each process group with the same number between different process groups as a new communication subdomain.

In this embodiment, it should be noted that each process in the new communication subdomain will be set with a new number. For example, process 0 in group 0 is process 0 in the new communication subdomain, process 0 in group 1 is process 1 in the new communication subdomain, process 0 in group 2 is process 2 in the new communication subdomain, and so on.

Operation A60, for each process in each communication subdomain, based on the number of elements and displacements corresponding to each division element in each process, performing data exchange on each division element in each process to obtain a target element in each process.

In this embodiment, the target elements include elements after data exchange and process-local elements without data exchange. Specifically, the following operations are performed for each process in each communication subdomain:

The number of data elements that need to be exchanged for message passing interface (MPI) communication exchange between processes in the next step is obtained to obtain an exchange quantity array. A corresponding exchange displacement array is generated according to the exchange quantity array, and then data exchange is performed on each communication subdomain based on the exchange quantity array and the exchange displacement array, to obtain the target elements in each of the processes. For example, process 0 in the new communication subdomain has data a0, a1, a2, a3, a4, a5, a6, a7, exchange quantity array counts is [2, 3, 3], exchange displacement array displs is [0, 2, 5]. That is, the first two (a0, a1) belong to process 0, (a2, a3, a4) are sent to process 1, and (a5, a6, a7) are sent to process 2.

Operation A70, merging and sorting the target element in each process to obtain a second sorting element.

In this embodiment, after the data exchange, the target elements in each process are merged and sorted to obtain the second sorting elements.

Operation A80, performing sorting by regular sampling on the second sorting element of each process in each communication subdomain in parallel to obtain each sorted gene subsequence.

In this embodiment, for each process in each communication subdomain, the second sorting element of each process in the communication subdomain is sorted by regular sampling in parallel to obtain each of the sorted gene subsequences.

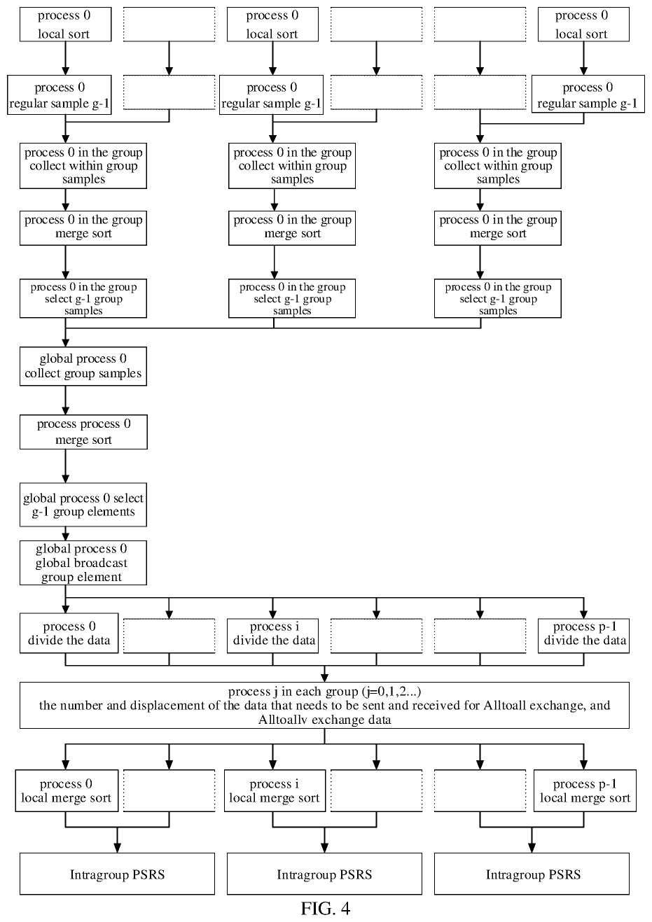

Further, as shown in FIG. 4, FIG. 4 is a schematic flowchart of a grouped parallel sorting by regular sampling in the genome assembly method of the present application. The number of processes is p, the process group is g, the group sample is the group sampling element, and the group element is the global sampling element. Firstly, for each element to be sorted in each process, each of the elements to be sorted is sorted to obtain a first sorting element, and then each process samples g−1 elements regularly in the first sorting element. The g−1 elements sampled by each process in the corresponding process group are collected through process 0 in the process group, g−1 elements are merged and sorted, and (g−1) elements after sorting are selected to obtain group sampling elements, then the group sampling elements of each process group are collected through the preset global process, the sampling elements of each group are sorted, and (g−1) elements after sorting are selected to obtain the group sampling elements. Then, globally broadcast the group sampling elements to each process by presetting the global process. Based on the group sampling elements, the first sorting elements in each process are divided to obtain each division element. Further, processes with the same number between different process groups are combined into a new communication subdomain, and each process in the new communication subdomain is renumbered, so as to exchange data in the new communication subdomain. In the process of data exchange, the number of data elements to be exchanged needs to be sent, and an exchange quantity array is obtained. A corresponding exchange displacement array according to the exchange quantity array is generated, and then data exchange is performed on each of the communication subdomains based on the exchange quantity array and the exchange displacement array, to obtain the target elements in each of the processes. Finally, the target elements in each process are merged and sorted to obtain the second sorting element. Finally, the second sorting elements of each process in each of the communication subdomains are sorted by regular sampling in parallel to obtain each of the sorting gene subsequences. As a result, the sampling points collected by process 0 are reduced from the original p*(p−1) to p/g*(g−1)+g*(g−1). That is, the complexity is reduced from O(1)(p\hat{ }2) to O(p+g\hat{ }2). For example, when the number of processes p is 16 and the process group g is 4 groups, the number of sampling points is reduced by 90%. When p>=24, g=4, the number of sampling points is reduced by at least 95%, and when p>=128, g=4, the number of sampling points is reduced by at least 99%.

In an embodiment of the present application, through the above solution, the MPI processes in the system are grouped by grouped parallel sorting by regular sampling, and the sampling number of each process is reduced from the original number of system processes to the number of process groups, which significantly reduces the number of system sampling points that increases quadratically due to the increase in the number of processes, and further reduces the time complexity of local sorting in the whole process of parallel sorting. In addition, by using packet communication instead of global communication, the number of sampling points is reduced, which not only reduces memory overhead, but also effectively reduces synchronization waiting time.

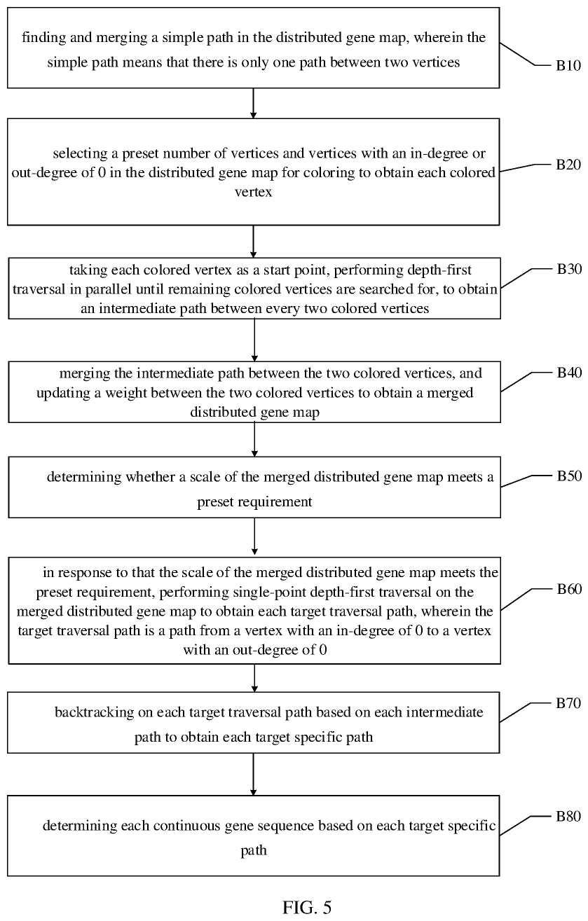

As shown in FIG. 5, based on the first embodiment of the present application, in another embodiment of the present application, the traversing the distributed gene map in parallel to obtain each continuous gene sequence includes:

Operation B10, finding and merging a simple path in the distributed gene map, the simple path means that there is only one path between two vertices.

In this embodiment, it is determined whether there is a start point of a simple path at the vertex of the distributed gene map. The simple path means that there is only one path between two vertices. If there is a start point of a simple path at the vertex of the distributed gene map, then based on the start point, traversing through the distributed gene map to find the end point corresponding to the simple path, merging the simple paths between the start point and the end point, and setting the weight between the start point and the end point to 1. It should be noted that merging simple paths means that if the path between two vertices is a simple path, an edge can be used instead of the simple path to connect the two vertices.

Operation B20, selecting a preset number of vertices and vertices with an in-degree or out-degree of 0 in the distributed gene map for coloring to obtain each colored vertex.

In this embodiment, color the vertices whose in-degree or out-degree is 0 in the distributed gene map and randomly select a preset number of vertices to obtain each colored vertex.

Operation B30, taking each colored vertex as a start point, performing depth-first traversal in parallel until remaining colored vertices are searched for, to obtain an intermediate path between every two colored vertices.

In this embodiment, each of the colored vertices is taken as a start point, to perform depth-first traversal in parallel in the distributed gene map, until any other colored vertex is searched by traversal, an intermediate path between every two colored vertices is obtained, and each intermediate path is saved.

Operation B40, merging the intermediate path between the two colored vertices, and updating a weight between the two colored vertices to obtain a merged distributed gene map.

In this embodiment, the intermediate paths between colored vertices are merged into one edge, to update the weight of the edge, for example, use the sum of the weights of the original edges of the intermediate path as the weight of the new edge.

Operation B50, determining whether a scale of the merged distributed gene map meets a preset requirement.

In this embodiment, the available memory of the system server is obtained, the space complexity of the depth-first search is calculated according to the graph scale of the merged distributed gene map, and whether the depth-first search can be performed on a single node is determined based on the space complexity.

Operation B60, in response to that the scale of the merged distributed gene map meets the preset requirement, performing single-point depth-first traversal on the merged distributed gene map to obtain each target traversal path, the target traversal path is a path from a vertex with an in-degree of 0 to a vertex with an out-degree of 0.

In this embodiment, it should be noted that the single-point depth-first traversal is a vertex with an in-degree of 0 in the figure as the start point for depth traversal. If it is satisfied, it proves that the graph size of the merged distributed gene map is small enough. A single-point depth-first traversal can be performed on the merged distributed gene map to obtain all paths between vertices with an in-degree of 0 and vertices with an out-degree of 0.

In addition, if not satisfied, return to the execution step: select a preset number of vertices and vertices with an in-degree or out-degree of 0 in the distributed gene map for coloring, and obtain each colored vertex until the graph scale meets the preset requirements.

Operation B70, backtracking on each target traversal path based on each intermediate path to obtain each target specific path.

Operation B80, determining each continuous gene sequence based on each target specific path.

In this embodiment, since intermediate paths between merged shading points are saved, when backtracking each of said target traversal paths, it is inquired whether there is a merged intermediate path between two vertices, thereby adding the intermediate traversal to the intermediate path to obtain each target specific path.

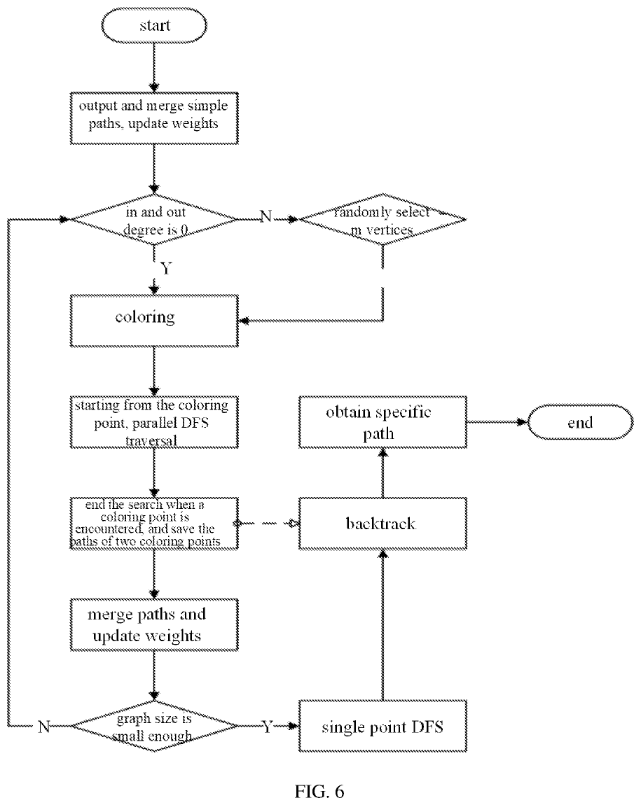

As shown in FIG. 6, FIG. 6 is a schematic flowchart of parallel traversal of the distributed gene map in the genome assembly method of the present application. m is the preset number, DFS is the depth-first traversal, and the specific path is the specific path of the panel. First, the simple paths are merged and the weights are updated, and then the vertices with an in-degree or out-degree of 0 are colored, and then m vertices are randomly selected for coloring to obtain multiple colored vertices. Starting from the colored vertex, the distributed gene map is traversed in parallel depth-first until another colored vertex is found, and the traversal is stopped. The intermediate path between the two colored vertices is saved, and then the intermediate paths of the two colored vertices are merged, and the weights are updated to obtain the merged distributed gene map. The space complexity of the depth traversal of the merged distributed gene map is calculated, and it is determined that the scale of the distributed gene map does not meet the preset requirements based on the space complexity. If the graph size of the merged distributed gene map does not meet the preset requirements, return to perform the operation: coloring the vertices with an in-degree or out-degree of 0, and then randomly selecting m vertices for coloring to continue merging distributed genes until the merged distributed genes meet the preset requirements. If the graph scale of the merged distributed gene map meets the preset requirements, performing single-point depth-first traversal on the merged distributed gene map to obtain each target traversal path, and based on the merged intermediate path, backtracking each of the target traversal paths to obtain each target specific path.

In an embodiment of the present application, through the above solution, by coloring the vertices with an in-degree or out-degree of 0, and then randomly selecting part of the vertices for coloring, the coloring vertex k-mer is selected as the seed for searching the contig of the continuous gene sequence, and extended backward in a single direction, which avoids the need to use additional synchronous communication algorithms to solve the situation that different processes search for the same continuous gene sequence contig. During the traversal process of the present application, when the next colored vertex is accessed, the depth search is stopped, the depth of a single depth-first search is effectively reduced, and the scale of the graph to be traversed is gradually reduced through graph coloring, thereby reducing the traversal time of each round of the distributed De Bruijn graph, and in the process of multi-level search traversal, by saving the merged intermediate path, the complete contigs of each continuous gene sequence can be traced back.

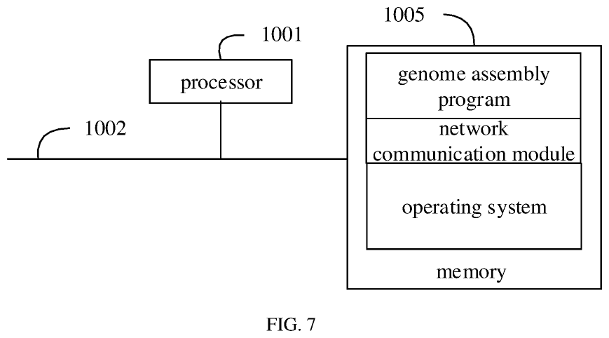

As shown in FIG. 7, FIG. 7 is a schematic structural diagram of a vehicle-mounted device for genome assembly in the hardware operating environment according to an embodiment of the present application.

As shown in FIG. 7, the vehicle-mounted device for genome assembly can include a processor 1001, such as a central processing unit (CPU), a memory 1005, and a communication bus 1002. The communication bus 1002 is configured to realize connection and communication between the processor 1001 and the memory 1005. The memory 1005 may be a high-speed random access memory (RAM), or a stable memory (non-volatile memory), such as a disk memory. The memory 1005 may also be a storage device independent of the aforementioned processor 1001.

In an embodiment, the vehicle-mounted device for genome assembly can also include a graphical user interface, a network interface, a camera, a radio frequency (RF) circuit, a sensor, an audio circuit, a WiFi module, and the like. The graphical user interface can include a display screen, an input sub-module such as a keyboard. The graphical user interface can also include a standard wired interface and a wireless interface. The network interface can include a standard wired interface and a wireless interface (such as a WIFI interface). Those skilled in the art should understand that the structure shown in FIG. 7 does not constitute a limitation on the vehicle-mounted device for genome assembly, and can include more or fewer components, a combination of some components, or differently arranged components than shown in the figure.

As shown in FIG. 7, the memory 1005 as a computer storage medium can include an operating system, a network communication module, and a genome assembly program. The operating system is a program that manages and controls the hardware and software resources of the vehicle-mounted device for genome assembly, and supports the operation of the genome assembly program and other software and/or programs. The network communication module is used to realize the communication between various components inside the memory 1005, and communicate with other hardware and software in the genome assembly device.

In the vehicle-mounted device for genome assembly shown in FIG. 7, the processor 1001 is configured to execute the genome assembly program stored in the memory 1005 to implement the operations of any one of the genome assembly methods described above.

The specific implementation of the vehicle-mounted device for genome assembly of the present application is basically the same as the embodiments of the genome assembly method as described above, and will not be repeated here.

In addition, the present application also provides a genome assembly apparatus, including:
an acquisition module configured for obtaining a gene short sequence, and determining a first segmentation value;
a segmentation module configured for segmenting the gene short sequence based on the first segmentation value to obtain each gene subsequence;
a global sorting module configured for globally sorting each gene subsequence based on a preset grouped parallel sorting by regular sampling to obtain each sorted gene subsequence;
a construction module configured for constructing a distributed gene map based on each sorted gene subsequence;
a parallel traversal module configured for traversing the distributed gene map in parallel to obtain each continuous gene sequence, and filling and assembling each continuous gene sequence to obtain each target continuous gene sequence; and
an assembly module configured for determining a second segmentation value, and in response to that the second segmentation value is greater than or equal to a preset maximum segmentation threshold, assembling each target continuous gene sequence to obtain a genome assembly result.

In an embodiment, the genome assembly apparatus is further configured for:
in response to that the second segmentation value is less than the preset maximum segmentation threshold, extracting each segmentation sequence from each target continuous gene sequence based on the second segmentation value;
segmenting the gene short sequence based on the second segmentation value to obtain each gene subsequence until obtaining each new sorted gene subsequence;
merging each segmentation sequence and each new sorted gene subsequence to obtain each merged gene sequence; and
constructing the distributed gene map based on each merged gene sequence to obtain new target continuous gene sequence until the determined segmentation value is greater than the preset maximum segmentation threshold, assembling each new target continuous gene sequence to obtain the genome assembly result.

In an embodiment, the segmentation module is further configured for:
adding the first segmentation value to the preset maximum segmentation threshold to obtain a segmentation window; and
scanning and segmenting the gene short sequence based on the segmentation window to obtain each gene subsequence, wherein a length of each gene subsequence is a length of the segmentation window.

In an embodiment, the global sorting module is further configured for:
reversing a prefix sequence corresponding to the first segmentation value in each gene subsequence and sorting in alphabetical order, and sorting each gene subsequence based on the sorting result to obtain each initial sorting sequence;
obtaining a number of processes, and grouping each process based on the number to obtain each process group, wherein each process in each process group is provided with a corresponding number;
using each initial sorting sequence as an element to be sorted, and assigning each element to be sorted to each process; and
performing sorting by regular sampling on each element to be sorted in parallel through each process in each process group to obtain each sorted gene subsequence.

In an embodiment, the global sorting module is further configured for:
for each element to be sorted in each process, sorting each element to be sorted to obtain a first sorting element, and performing regular sampling on the first sorting element to obtain a first sampled element;
sending the first sampled element in each process to a first numbered process of the corresponding process group, for the first numbered process in each process group, sorting and performing regular sampling on each first sampled element in parallel to obtain group sampling elements of each process group;
sending each group sampling element to a preset global process, and sorting and performing regular sampling on each group sampling element through the preset global process to obtain a global sampling element;
dividing the first sorting element in each process based on the global sampling element to obtain each division element, and recording number of elements and displacements corresponding to each division element;
forming each process group with the same number between different process groups as a new communication subdomain;
for each process in each communication subdomain, based on the number of elements and displacements corresponding to each division element in each process, performing data exchange on each division element in each process to obtain a target element in each process;
merging and sorting the target element in each process to obtain a second sorting element; and
performing sorting by regular sampling on the second sorting element of each process in each communication subdomain in parallel to obtain each sorted gene subsequence.

In an embodiment, the construction module is further configured for:
merging the same sorted gene subsequence, and counting a frequency of each sorted gene subsequence;
determining each target sorted gene subsequence whose frequency exceeds a preset frequency threshold; and
taking each target sorted gene subsequence as an edge of the distributed gene map, and using a sequence whose prefix length is the first segmentation value in each target sorted gene subsequence as a vertex of the distributed gene map.

In an embodiment, the parallel traversal module is further configured for:
finding and merging a simple path in the distributed gene map, wherein the simple path means that there is only one path between two vertices;
selecting a preset number of vertices and vertices with an in-degree or out-degree of 0 in the distributed gene map for coloring to obtain each colored vertex;
taking each colored vertex as a start point, performing depth-first traversal in parallel until remaining colored vertices are searched for, to obtain an intermediate path between every two colored vertices;
merging the intermediate path between the two colored vertices, and updating a weight between the two colored vertices to obtain a merged distributed gene map;
determining whether a scale of the merged distributed gene map meets a preset requirement;
in response to that the scale of the merged distributed gene map meets the preset requirement, performing single-point depth-first traversal on the merged distributed gene map to obtain each target traversal path, wherein the target traversal path is a path from a vertex with an in-degree of 0 to a vertex with an out-degree of 0;
backtracking on each target traversal path based on each intermediate path to obtain each target specific path; and
determining each continuous gene sequence based on each target specific path.

In an embodiment, the genome assembly apparatus is further configured for:
aligning the gene short sequence with each continuous gene sequence; and
filling and assembling each continuous gene sequence based on the alignment result to obtain each target continuous gene sequence.

The specific implementation of the genome assembly apparatus of the present application is basically the same as the embodiments of the genome assembly method as described above, and will not be repeated here.

The embodiment of the present application provides a storage medium. The storage medium is a computer-readable storage medium, and the computer-readable storage medium stores one or more programs. The one or more programs can also be executed by one or more processors to implement the operations of any one of the genome assembly methods described above.

The specific implementation of the computer-readable storage medium of the present application is basically the same as the embodiments of the genome assembly method as described above, and will not be repeated here.

The above are only some embodiments of the present application, and do not limit the scope of the present application thereto. Under the concept of the present application, equivalent structural transformations made according to the description and drawings of the present application, or direct/indirect application in other related technical fields are included in the scope of the present application.

What is claimed is:

1. A genome assembly method, comprising:
obtaining a gene short sequence, and determining a first segmentation value;
segmenting the gene short sequence based on the first segmentation value to obtain each gene subsequence;
globally sorting each gene subsequence based on a preset grouped parallel sorting by regular sampling to obtain each sorted gene subsequence, wherein the preset grouped parallel sorting by regular sampling is an algorithm that performs sorting by regular sampling on each gene subsequence in parallel based on each process after pre-grouping;
constructing a distributed gene map based on each sorted gene subsequence;
traversing the distributed gene map in parallel to obtain each continuous gene sequence, and filling and assembling each continuous gene sequence to obtain each target continuous gene sequence; and
determining a second segmentation value, and in response to that the second segmentation value is greater than or equal to a preset maximum segmentation threshold, assembling each target continuous gene sequence to obtain a genome assembly result.

2. The genome assembly method according to claim 1, wherein after the determining the second segmentation value, the genome assembly method further comprises:
in response to that the second segmentation value is less than the preset maximum segmentation threshold, extracting each segmentation sequence from each target continuous gene sequence based on the second segmentation value;
segmenting the gene short sequence based on the second segmentation value to obtain each gene subsequence until obtaining each new sorted gene subsequence;
merging each segmentation sequence and each new sorted gene subsequence to obtain each merged gene sequence; and
constructing the distributed gene map based on each merged gene sequence to obtain new target continuous gene sequence until the determined segmentation value is greater than the preset maximum segmentation threshold, assembling each new target continuous gene sequence to obtain the genome assembly result.

3. The genome assembly method according to claim 1, wherein the segmenting the gene short sequence based on the first segmentation value to obtain each gene subsequence comprises:
adding the first segmentation value to the preset maximum segmentation threshold to obtain a segmentation window; and
scanning and segmenting the gene short sequence based on the segmentation window to obtain each gene subsequence, wherein a length of each gene subsequence is a length of the segmentation window.

4. The genome assembly method according to claim 3, wherein the globally sorting each gene subsequence based on the preset grouped parallel sorting by regular sampling to obtain each sorted gene subsequence comprises:
reversing a prefix sequence corresponding to the first segmentation value in each gene subsequence and sorting in alphabetical order, and sorting each gene subsequence based on the sorting result to obtain each initial sorting sequence;
obtaining a number of processes, and grouping each process based on the number to obtain each process group, wherein each process in each process group is provided with a corresponding number;
using each initial sorting sequence as an element to be sorted, and assigning each element to be sorted to each process; and
performing sorting by regular sampling on each element to be sorted in parallel through each process in each process group to obtain each sorted gene subsequence.

5. The genome assembly method according to claim 4, wherein the performing sorting by regular sampling on each element to be sorted in parallel through each process in each process group to obtain each sorted gene subsequence comprises:
for each element to be sorted in each process, sorting each element to be sorted to obtain a first sorting element, and performing regular sampling on the first sorting element to obtain a first sampled element;
sending the first sampled element in each process to a first numbered process of the corresponding process group, for the first numbered process in each process group, sorting and performing regular sampling on each first sampled element in parallel to obtain group sampling elements of each process group;
sending each group sampling element to a preset global process, and sorting and performing regular sampling on each group sampling element through the preset global process to obtain a global sampling element;
dividing the first sorting element in each process based on the global sampling element to obtain each division element, and recording number of elements and displacements corresponding to each division element;
forming each process group with the same number between different process groups as a new communication subdomain;
for each process in each communication subdomain, based on the number of elements and displacements corresponding to each division element in each process, performing data exchange on each division element in each process to obtain a target element in each process;
merging and sorting the target element in each process to obtain a second sorting element; and
performing sorting by regular sampling on the second sorting element of each process in each communication subdomain in parallel to obtain each sorted gene subsequence.

6. The genome assembly method according to claim 1, wherein the constructing the distributed gene map based on each sorted gene subsequence comprises:
merging the same sorted gene subsequence, and counting a frequency of each sorted gene subsequence;
determining each target sorted gene subsequence whose frequency exceeds a preset frequency threshold; and
taking each target sorted gene subsequence as an edge of the distributed gene map, and using a sequence whose prefix length is the first segmentation value in each target sorted gene subsequence as a vertex of the distributed gene map.

7. The genome assembly method according to claim 1, wherein the traversing the distributed gene map in parallel to obtain each continuous gene sequence comprises:
finding and merging a simple path in the distributed gene map, wherein the simple path means that there is only one path between two vertices;
selecting a preset number of vertices and vertices with an in-degree or out-degree of 0 in the distributed gene map for coloring to obtain each colored vertex;
taking each colored vertex as a start point, performing depth-first traversal in parallel until remaining colored vertices are searched for, to obtain an intermediate path between every two colored vertices;
merging the intermediate path between the two colored vertices, and updating a weight between the two colored vertices to obtain a merged distributed gene map;
determining whether a scale of the merged distributed gene map meets a preset requirement;
in response to that the scale of the merged distributed gene map meets the preset requirement, performing single-point depth-first traversal on the merged distributed gene map to obtain each target traversal path, wherein the target traversal path is a path from a vertex with an in-degree of to a vertex with an out-degree of 0;
backtracking on each target traversal path based on each intermediate path to obtain each target specific path; and
determining each continuous gene sequence based on each target specific path.

8. The genome assembly method according to claim 1, wherein the filling and assembling each continuous gene sequence to obtain each target continuous gene sequence comprises:
aligning the gene short sequence with each continuous gene sequence; and
filling and assembling each continuous gene sequence based on the alignment result to obtain each target continuous gene sequence.

9. A vehicle-mounted device for genome assembly, comprising:
a memory;
a processor; and
a genome assembly program stored in the memory,
wherein when the genome assembly program is executed by the processor, the genome assembly method according to claim 1 is implemented.

10. A non-transitory computer-readable storage medium, wherein a genome assembly program is stored in the non-transitory computer-readable storage medium, and when the genome assembly program is executed by a processor, the genome assembly method according to claim 1 is implemented.
